(19) United States
(12) Patent Application Publication
(10) Pub. No.: US 2025/0049356 A1
(43) Pub. Date: Feb. 13, 2025

(54) SYSTEM AND METHOD FOR FUSING RF SPO2 MEASUREMENTS WITH OPTICAL SPO2 MEASUREMENTS

(71) Applicant: Know Labs, Inc., Seattle, WA (US)

(72) Inventor: John CRONIN, Williston, VT (US)

(21) Appl. No.: 18/798,085

(22) Filed: Aug. 8, 2024

Related U.S. Application Data
(60) Provisional application No. 63/518,224, filed on Aug. 8, 2023.

(51) Int. Cl. A61B 5/1455; A61B 5/05; G16H 50/70

(52) U.S. Cl. CPC A61B 5/14551; A61B 5/05; G16H 50/70

(57) ABSTRACT

A system that includes a real-time, non-invasive radio frequency (RF) device for detecting analytes, such as SPO2, in a patient's blood. The RF device detects a wave signal that results from the transmission of RF waves into the patient's body. The wave signal is compared to known standard waveforms, and similar waveforms are input into a machine learning algorithm in order to determine one or more health parameters of the person. Health parameters are collected from an optical SPO2 device, stored, and fused with the health parameters from the RF device. The system then notifies the person and/or health professionals of the person's health status.

This application claims the benefit of U.S. Provisional Application No. 63/518,224, filed Aug. 8, 2023, which application is incorporated herein by reference in its entirety.

### FIELD

The present disclosure is generally related to systems and methods of monitoring health parameters and, more particularly, relates to a system and a method of monitoring real-time SPO2 levels using radio frequency signals.

### BACKGROUND

One problem affecting the accuracy of SpO2 measurements using pulse oximeters is the presence of motion artifacts, which occur when movement or shaking interferes with the readings, particularly in agitated, restless, or uncooperative patients.

Another issue impacting the reliability of SpO2 measurements is poor peripheral perfusion, which can occur due to hypotension, vasoconstriction, or other factors, leading to inaccurate readings as the pulse oximeter fails to properly detect changes in light absorption by hemoglobin in the blood.

Lastly, factors such as skin pigmentation, the use of dark nail polish, and carbon monoxide poisoning can also negatively influence the accuracy of SpO2 measurements, as they interfere with the pulse oximeter's ability to effectively detect changes in the absorption of light by hemoglobin in the blood.

Pulse oximeters are widely used as a reliable and non-invasive method for assessing oxygen saturation levels (SpO2). However, there are potential problems that can affect the accuracy of these measurements.

### BRIEF DESCRIPTION OF DRAWINGS

FIG. 1: Illustrates a system for radio frequency health monitoring, according to an embodiment.

FIG. 2: Illustrates an example operation of a Device Base Module, according to an embodiment.

FIG. 3: Illustrates an example operation of an Input Waveform Module, according to an embodiment.

FIG. 4: Illustrates an example operation of a Matching Module, according to an embodiment.

FIG. 5: Illustrates an example operation of a Machine Learning Module, according to an embodiment.

FIG. 6: Illustrates an example operation of a Connection Module, according to an embodiment.

FIG. 7: Illustrates an example operation of a Data Fusion Module, according to an embodiment.

FIG. 8: Illustrates an example operation of a Report Module, according to an embodiment.

FIG. 9: Illustrates an example of a Fusion Example Database, according to an embodiment.

FIG. 10: Illustrates an example operation of an Error Module, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

U.S. Pat. Nos. 10,548,503, 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870,U.S. 2022/0151553, are each individually incorporated herein by reference in its entirety.

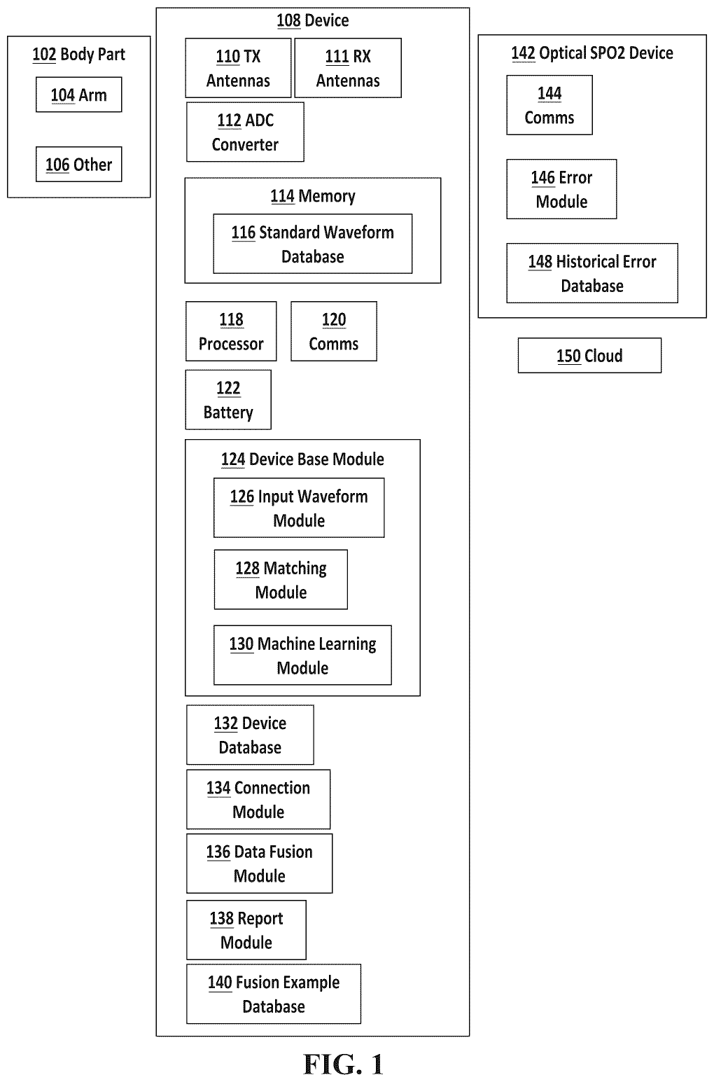

FIG. 1 is a schematic illustration of a system for radio frequency health monitoring. This system is configured to be attached to or in proximity to a body part 102. The body part 102 may be an arm 104.

The body part 102 may be another body part 106 besides an arm, such as a leg, finger, chest, head, or any other body part from which useful medical parameters can be taken.

The system may further comprise a device 108, which may be a wearable and portable device such as, but not limited to, a cell phone, a smartwatch, a tracker, a wearable monitor, a wristband, and a personal blood monitoring device.

The system may further comprise a set of TX antennas 110. TX antennas 110 may be configured to transmit RF in the RF Activated Range from 500 MHz to 300 GHz. In one embodiment, a pre-defined frequency may correspond to a range suitable for the human body. For example, the one or more TX antennas 110 would use radio frequency signals at a range of 120-126 GHz.

The system may further comprise a set of RX antennas 111. The one or more RX antennas 111 may be configured to receive the RF signals in response to the TX RF signal.

The system may further comprise an ADC converter 112, which may be configured to convert the RF signals from an analog signal into a digital processor readable format.

The system may further comprise memory 114, which may be configured to store the transmitted RF signals by the one or more TX antennas 110 and receive a portion of the transmitted RF signals from the one or more RX antennas 111. Further, the memory 114 may also store the converted digital processor readable format by the ADC converter 112. The memory 114 may include suitable logic, circuitry, and/or interfaces that may be configured to store a machine code and/or a computer program with at least one code section executable by the processor 118. Examples of implementation of the memory 114 may include, but are not limited to, Random Access Memory (RAM), Read Only Memory (ROM), Hard Disk Drive (HDD), and/or a Secure Digital (SD) card.

The system may further comprise a standard waveform database 116, which may contain standard waveforms for known patterns. These may be raw or converted device readings from patients or persons with known conditions. For example, the standard waveform database 116 may include raw or converted device readings from a patient known to have diabetes or an average of multiple patients. This data can be compared to readings from a person with an unknown condition in order to determine if the waveforms taken from that person match any of the known standard waveforms.

The system may further comprise a processor 118, which may facilitate the operation of the device 108 according to the instructions stored in the memory 114. The processor 118 may include suitable logic, circuitry, interfaces, and/or code that may be configured to execute a set of instructions stored in the memory 114.

The system may further comprise device comms 120, which may communicate with a network. Examples of networks may include but are not limited to, the Internet, a cloud network, a Wireless Fidelity (Wi-Fi) network, a Wireless Local Area Network (WLAN), a Local Area Network (LAN), Long Term Evolution (LTE), and/or a Metropolitan Area Network (MAN).

The system may further comprise a battery 122, which may power hardware modules of the device 108. The device 108 may be configured with a charging port to recharge the battery 122. Charging of the battery 122 may be wired or wireless.

The system may further comprise a device base module 124, which may be configured to store instructions for executing the computer program from the converted digital processor readable format of the ADC converter 112.

The device base module 124 may be configured to facilitate the operation of the processor 118, the memory 114, the TX antennas 110, RX antennas 111, and the comms 120. Further, the device base module 124 may be configured to create polling of the RF Activated Range from 500 MHz to 300 GHz. It can be noted that the device base module 124 may be configured to filter the RF Activated Range from 500 MHz to 300 GHz received from one or more RX antennas 111.

The system may further comprise an input waveform module 126, which may extract a radio frequency waveform from memory. This may be the raw or converted data recording from the RX antennas 111 from a patient wearing the device. If the entire radio frequency is too long for effective matching, the input waveform module 126 may select a time interval within the data set. This input waveform may then be sent to the matching module 128.

The system may further comprise a matching module 128, which may match the input waveform and each of the standard waveforms in the standard waveform database 116 by performing a convolution and/or cross-correlation or other matching technique of the input waveform and the standard waveform. These convolutions and/or cross-correlations are then sent to the machine learning module.

The system may further comprise a machine learning module 130 which has been trained to identify health parameters based on the convolution and/or cross-correlations of the input and standard waveforms. The machine learning module 130 receives the convolutions and cross-correlations from the matching module 128 and outputs any health parameters identified.

The system may further comprise a device database 132, which may store the health parameters output by the machine learning module 130.

The system may further comprise a connection module 134, which may connect to the optical SPO2 device 142 and collect data which then may be stored in the device database 132.

The system may further comprise a data fusion module 136, which may be configured to fuse the real-time analyte data from the device 108, including SPO2 measurements and other analytes, and the collected data from the optical SPO2 device 142 stored in the device database 132. The data fusion module 136 may employ suitable algorithms, logic, circuitry, and/or interfaces to effectively perform the data fusion process. Fusing data may refer to the process of integrating multiple sources of information or data sets into a single, coherent output. This process may involve aligning, synchronizing, and combining data from different sources while maintaining accuracy, consistency, and integrity. The system may further comprise a report module 138, which may display a report of the fused data from the data fusion module 136. This report may be sent to an email address, displayed on a screen, sent to another module, or otherwise reported.

The system may further comprise a fusion example database 140, which may contain examples illustrating various issues encountered during the measurement of analytes using different devices, along with the corresponding data fusion methods employed to resolve these issues. The fusion example database 140 may serve as a resource for facilitating the identification of optimal data fusion techniques when analyzing information obtained from disparate devices. Each entry within the database may comprise A brief description of the issue encountered during the measurement of analytes using different devices, a detailed explanation of the data fusion technique utilized to resolve the issue described, example data from the device 108 and optical SPO2 device 142, and an example of the fused data.

The system may further comprise an optical SPO2 device 142, which may be configured to non-invasively measure the oxygen saturation level in a user's blood. The optical SPO2 device 142 may utilize a combination of light-emitting diodes (LEDs) and a photodetector to detect the absorption of light by hemoglobin in the blood, thereby determining the oxygen saturation level. The optical SPO2 device 142 may be adapted to be worn on various body parts, such as a fingertip, earlobe, or forehead, to facilitate accurate and reliable measurements. The data collected by the optical SPO2 device 142 may be transmitted to other system components, such as the connection module 134, for further processing and analysis. The optical SPO2 device 142 may be calibrated periodically to ensure accurate and consistent readings. Furthermore, the optical SPO2 device 142 may be designed with suitable logic, circuitry, interfaces, and/or code to comply with regulatory requirements and data privacy standards, ensuring the protection of sensitive user information.

The system may further comprise an optical SPO2 device comms 144, which may be a communication element such as a Wi-Fi or RFC transmitter that can send data from the optical SPO2 device 142 to the connection module 134 of the device 108.

The system may further comprise an error module 146, which may compare the waveform recordings from the optical SPO2 device 142 to waveforms in the historical error database 148 to determine any known errors in the data. For example, data from the optical SPO2 device 142 may contain motion artifacts if a patient moves. These artifacts can be identified by comparing the data to data in the historical error database 148, which also contains motion artifacts, and identifying that the source of error is the patient's motion. If the type of error is known, such as motion, poor tissue perfusion, dark skin pigmentation, etc., that information may be sent to the data fusion module 136, which may use the information to determine which method of data fusion to perform.

The system may further comprise a historical error database 148, which may contain waveforms for known error patterns. These may be optical SPO2 device 142 readings from patients or persons with known errors in the data. For example, the historical error database 148 may include readings from patients that were moving, causing motion artifacts in the data, or patients wearing dark nail polish, causing poor optical transparency and low accuracy readings.

The system may further comprise a cloud 150 or communication network, which may be wired and/or wireless. The communication network, if wireless, may be implemented using communication techniques such as Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art. The communication network may allow ubiquitous access to shared pools of configurable system resources and higher-level services that can be rapidly provisioned with minimal management effort, often over the Internet and relies on sharing of resources to achieve coherence and economics of scale, like a public utility, while third-party clouds enable organizations to focus on their core businesses instead of expending resources on computer infrastructure and maintenance.

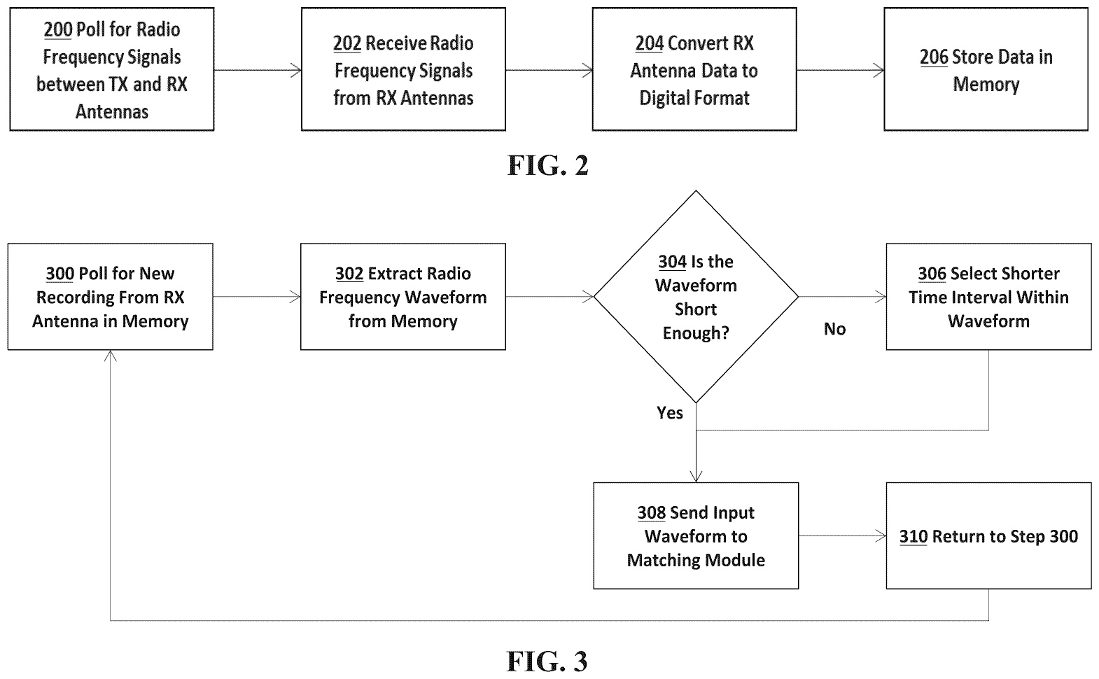

FIG. 2 displays an example operation of the device base module 124. The process may begin with the device base module 124 polling the Active Range RF signals between the one or more TX antennas 110 and the one or more RX antennas 111 at step 200. The device base module 124 may be configured to read and process instructions stored in the memory 114 using the processor 118. The TX antennas 110 may be configured to transmit RF in the RF Activated Range from 500 MHz to 300 GHz. For example, the one or more TX antennas 110 may use RF signals at a range of 500 MHz to 300 GHz. The device base module 124 may receive the RF frequency signals from the one or more RX antennas 111 at step 202. For example, an RX antenna 111 receives an RF of frequency range 300-330 GHz from the patient's blood. The device base module 124 may be configured to convert the received RF signals into a digital format using the ADC 112 at step 204. For example, the received RF of frequency range 300-330 GHz is converted into a 10-bit data signal. The device base module 124 may be configured to store converted digital format into the memory 114 at step 206.

FIG. 3 illustrates an example operation of the input waveform module 126. The process may begin with the input waveform module 126 polling, at step 300, for newly recorded data from the RX antennas 111 stored in memory 114. The input waveform module 126 may extract, at step 302, the recorded radio frequency waveform from memory. If there is more than one waveform recorded, the input waveform module 126 may select each waveform separately and loop through the following steps. The input waveform module 126 may determine, at step 304, if the waveform is small enough to be an input waveform for the matching module 128. This will depend on the computational requirements and/or restrictions of the matching module 128. If the waveform is short enough, the input waveform module 126 may skip to step 308. If the waveform is too long, the input waveform module 126 may select, at step 306, a shorter time interval within the entire recorded waveform. For example, if the waveform is 5 minutes long, then only a 30-second interval may be selected. The interval may be selected at random or by a selection process. The input waveform module 126 may send, at step 308, the input waveform to the matching module 128. The input waveform module 126 may return, at step 310, to step 300.

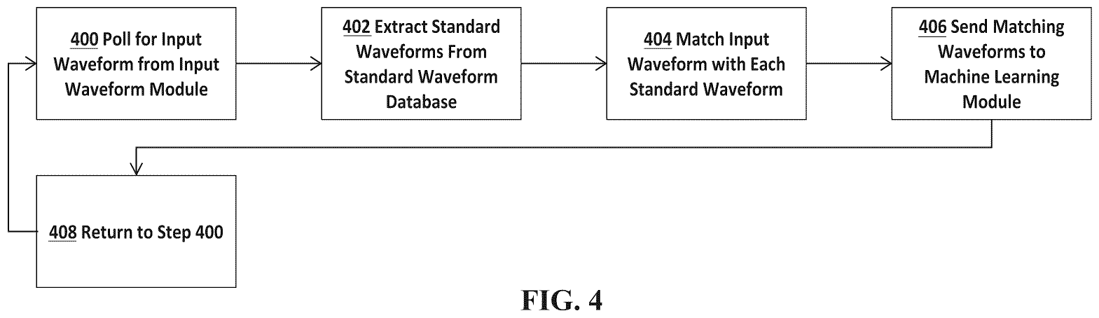

FIG. 4 illustrates an example operation of the matching module 128. The process may begin with the matching module 128 polling, at step 400, for an input waveform from the input waveform module 126. The matching module 128 may extract, at step 402, each standard waveform from the standard waveform database 116. The matching module 128 may match, at step 404, the input waveform with each standard waveform. Matching may be determining which standard waveforms the input waveform is similar to. Matching may involve convolution and/or cross-correlation of the waveforms or any other matching technique. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two wave functions, the output will give a similarity value between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function whose values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the standard waveform matches the input waveform. Matching standard waveforms, the input waveform, the cross-correlation of both, and/or the convolution of both may be used as an input to the machine learning algorithm of the machine learning module 130. The matching module 128 may send, at step 406, the matching waveforms to the machine learning module 130. Matching waveforms may refer to the standard waveforms that were similar to the input waveform, the waveforms that were generated via convolution and/or cross-correlation, or both. The matching module 128 may return, at step 408, to step 400.

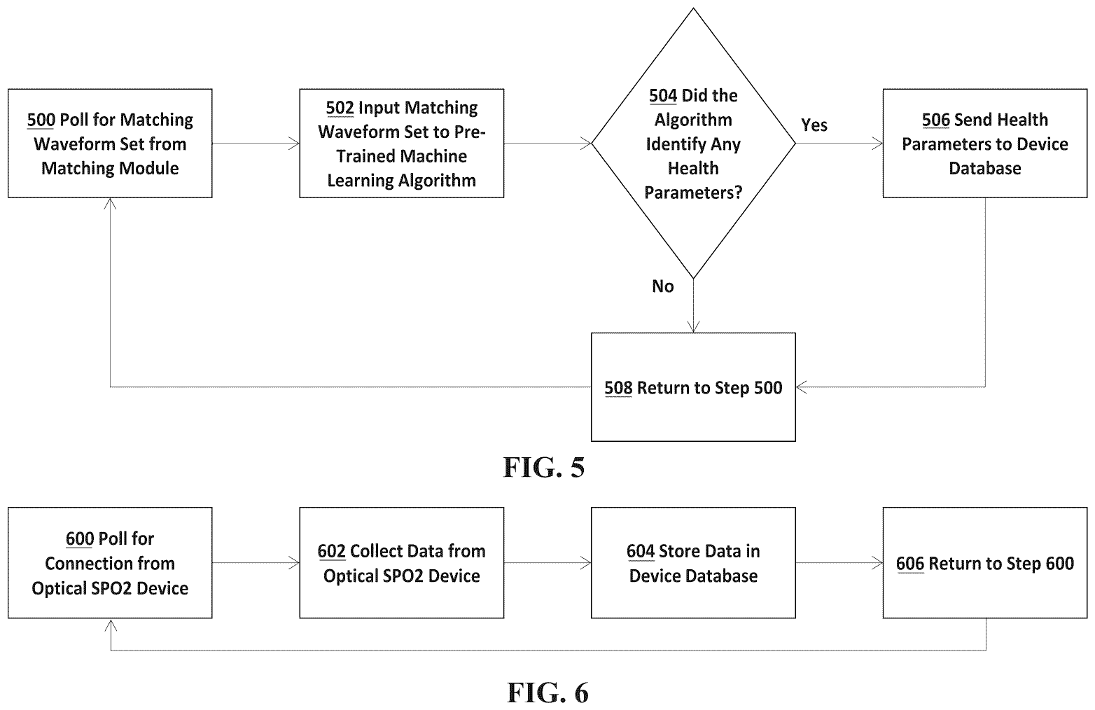

FIG. 5 illustrates an example operation of the machine learning module 130. The process may begin with the machine learning module 130 polling, at step 500, for a set of matching waveforms from the matching module 128. Matching waveforms may be a set of standard waveforms that are similar to the input waveform or statistical combinations of the input waveform and standard waveforms, such as convolutions or cross-correlations. The machine learning module 130 may input, at step 502, the set of received waveforms into a pre-trained machine learning algorithm. The machine learning algorithm may be trained on similar sets of matched waveforms where the input waveform is from a patient whose health parameters are known. The waveforms, such as a set of X and Y values, may be input directly into the algorithm. The matching waveforms may each be summarized as a closest fit function or may be transformed into a set of sine waves using a Fourier transform. Training data should be labeled with the correct output, such as the type of waveform. In order to prepare the data, the waveforms need to be processed and converted into a format that can be used by the algorithm. Once the data is prepared, the algorithm is trained on the labeled data. The model uses this data to learn the relationships between the waveforms and their corresponding outputs. During the training process, the model will adjust its parameters to minimize the error between its predictions and the correct outputs. Once the model has been trained and fine-tuned, it can be used to recognize waveforms in new, unseen data. This could be done by giving the input waveforms, then the algorithm will predict the health parameters. The machine learning module 130 may determine, at step 504, if the algorithm identified any health parameters. Identification may require a certain interval of confidence. For example, if the machine learning algorithm determines that it is more than 70% likely that a health parameter is correct, then that parameter may be considered identified. If multiple conflicting parameters exist, then the most confident may be used. For example, if the algorithm determines that it is 75% likely that the patient's SPO2 level is between 91% and 92% and 90% likely that the patient's SPO2 is between 92% and 93%, then the more confident value of 92% to 93% may be identified. If none of the results from the machine learning algorithm are above the confidence threshold, or the results are otherwise inconclusive, the machine learning module 130 may skip to step 508. If any health parameters were identified, the machine learning module 130 may send, at step 506, the health parameters to the device database 132. The machine learning module 130 may return, at step 508, to step 500.

FIG. 6 illustrates an example operation of the connection module 134. The connection module 134 polls, at step 600, for a connection from the optical SPO2 device 142. If no connection is detected, the connection module 134 may search for an optical SPO2 device attempting to connect. The connection module 134 may collect, at step 602, data from the optical SPO2 device. This may be real-time data or periodically updated data. The connection module 134 may store, at step 604, the data from the optical SPO2 device 142 in the device database 132. The connection module 134 may add contextual data such as the ID of the optical SPO2 device, type of optical SPO2 device, time data was retrieved, etc., to the received data before storage in the device database 132. The connection module 134 may return, at step 606, to step 600.

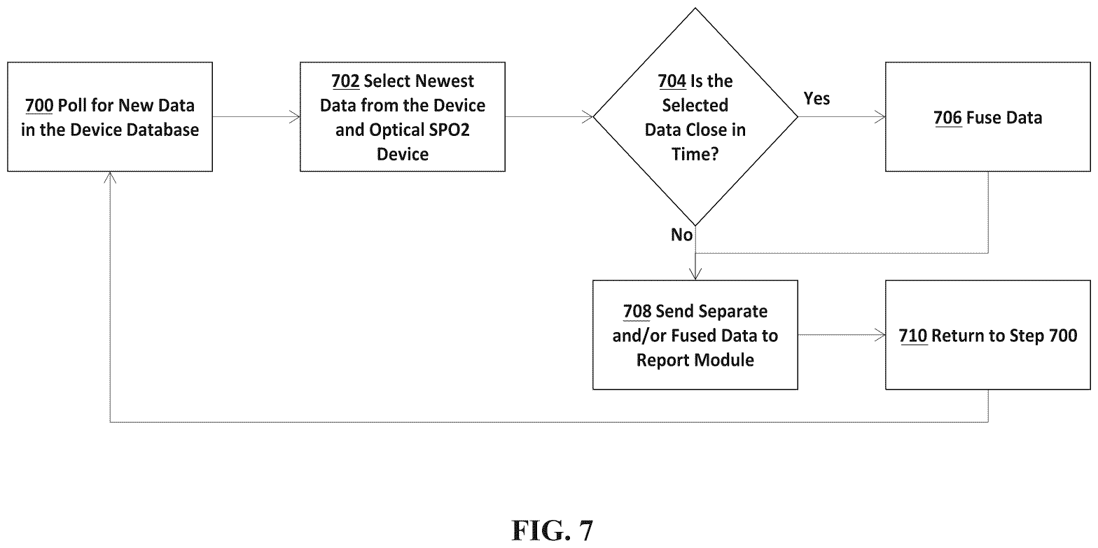

FIG. 7 illustrates an example operation of the data fusion module 136. The data fusion module 136 may poll, at step 700, for new data in the device database. This new data may be data from the device 108 or from the optical SPO2 device 142. The data fusion module 136 may select, at step 702, the newest data from the device 108 and from the optical SPO2 device 142. This may be a single data point or a set of data. The data fusion module 136 may determine, at step 704, if the selected data is close in time. Close in time may mean each piece or set of data is timestamped within a few seconds of each other. For example, the newest data from the device 108 may be real-time, but the newest reading from the optical SPO2 device may have been 10 minutes ago. This data would not be close in time if the threshold is a few seconds. The threshold may be varied based on the health parameter being measured. For example, if the optical SPO2 device is recording sleep cycle activity, then recordings from 10 minutes ago may be relevant because of the gradual nature of sleep cycles, but if the optical SPO2 device is recording heart rate, then data from minutes ago may be highly inaccurate. The time threshold for what data is considered close in time may be set by the user of the device, manufacturer of the device, and/or another module. If the selected data is not close in time, the data fusion module may skip to step 708. The data fusion module 136 may fuse, at step 706, the selected data from the device 108 and optical SPO2 device 142. The fusion of data may encompass multiple processes, with the chosen process contingent upon the types of data being compared. For instance, if both pieces or sets of data represent measurements of the same parameter, such as SPO2, then fusing the data may involve aligning and combining data to ensure its accuracy and consistency. Aligning could entail calculating the mean or median value. The data might be enhanced, with an error returned if both measurements fall outside a predetermined normal range. For example, a patient is wearing both an RF-based and an optical SPO2 device. The RF-based device records an SPO2 value of 95%, while the optical device records a value of 94%. The data fusion module calculates the mean value of the two measurements (94.5%) and uses this value as the final, combined SPO2 reading. Fused data could represent a correlation function of two sets of data, such as correlating sleep cycle measurements from the device 108 with SPO2 readings from the optical SPO2 device 142 to monitor the influence of oxygen saturation on sleep quality. For example, a patient is monitored during sleep, and their SPO2 readings from the optical SPO2 device 142 are compared with sleep cycle data from the device 108. The data fusion module identifies that the patient's oxygen saturation levels drop significantly during the REM sleep stage, indicating a possible correlation between sleep quality and oxygen saturation levels. Fusing the data may involve adjusting one piece or set of data based on the other. For instance, pulse rate readings from the device 108 could be employed to adjust SPO2 readings, which are calibrated for a standard pulse rate. Data fusion may include monitoring oxygen saturation levels during periods of motion or low perfusion and selecting the data from the source with the least interference. For example, A patient is exercising while wearing both a device 108 and an optical SPO2 device 142. The patient's motion affects the optical SPO2 device's 140 readings, leading to inaccurate results. The data fusion module 136 combines the readings from both devices, giving more weight to the device 108 data to produce a more accurate SPO2 reading during the exercise. The fusion process can also help assess the accuracy of either device by comparing the data from both devices to identify discrepancies in the readings. For example, a patient is monitored using both the device 108 and the optical SPO2 device 142. The data fusion module compares the readings from both devices and identifies that the optical SPO2 device 142 consistently reports lower SPO2 values. This information helps the medical team determine that the optical SPO2 device 142 or the device 108 may require recalibration. Data fusion may involve patient or context information. For example, if the patient's skin contains high amounts of melanin, the optical SPO2 device 142 may be unreliable, but the device 108 may be unaffected. The data fusion module 136 combines the readings from both devices, giving more weight to the device 108 data to produce a more accurate SPO2 reading for that patient. The data fusion module 136 may send, at step 708, each piece or set of data and/or the fused data, if available, to the report module 138. The data fusion module 136 may send only the most up-to-date set of data. For example, if the device 108 is not recording any data due to the recording components being turned off or broken, then only the data from the optical SPO2 device 142 may be sent to the report module 138. The data fusion module 136 may return, at step 710, to step 700.

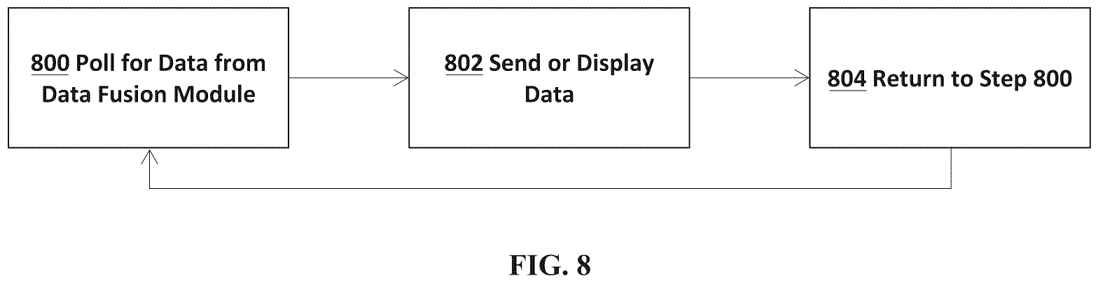

FIG. 8 displays an example operation of the report module 138. The process may begin with the report module 138 polling, at step 800, for data from the data fusion module 136. The report module 138 may send and/or display, at step 802, the received data. If a display is connected to the device 108 or can be reached by other means, the report module 138 may display the data directly. If not, the report module may send data in real-time or periodically. Data may be sent to an email address, a database, a module, or any other destination. The data may be sent to and/or displayed at multiple locations. The report module 138 may return, at step 804, to step 800.

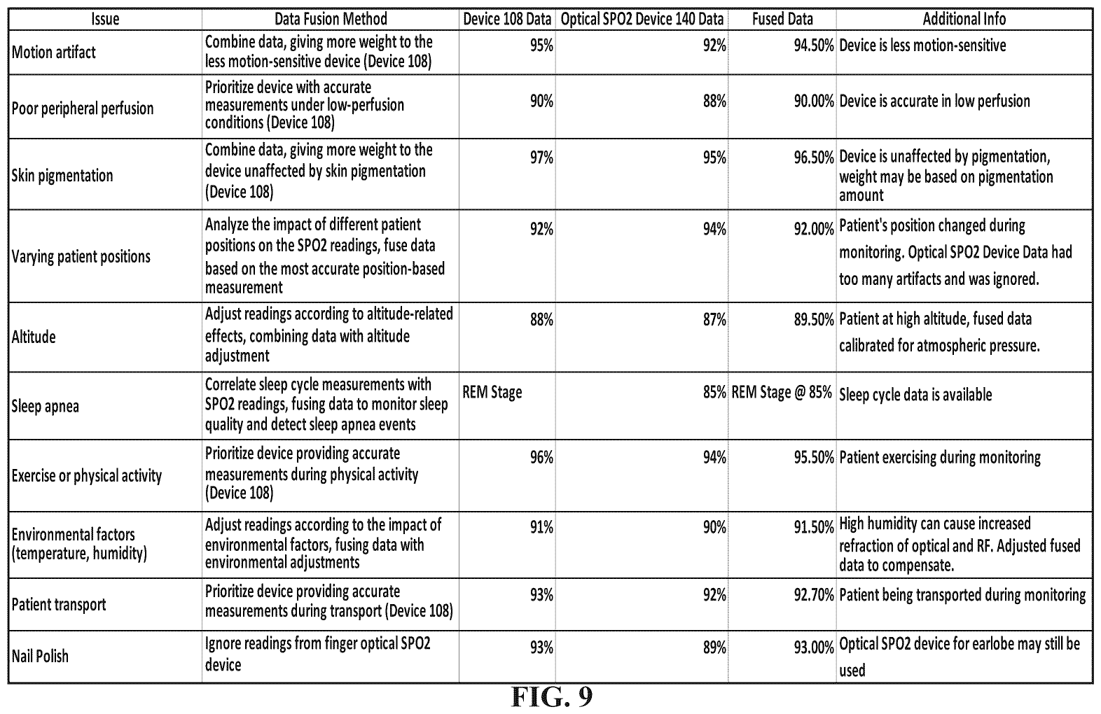

FIG. 9 illustrates a fusion example database 140. The fusion example database 140 contains examples illustrating various issues encountered during the measurement of analytes using different devices, along with the corresponding data fusion methods employed to resolve these issues. The fusion example database 140 may serve as a resource for facilitating the identification of optimal data fusion techniques when analyzing information obtained from disparate devices. Each entry within the database may comprise a brief description of the issue encountered during the measurement of analytes using different devices; a detailed explanation of the data fusion technique utilized to resolve the issue described; example data from the device 108 and optical SPO2 device 142; and an example of the fused data.

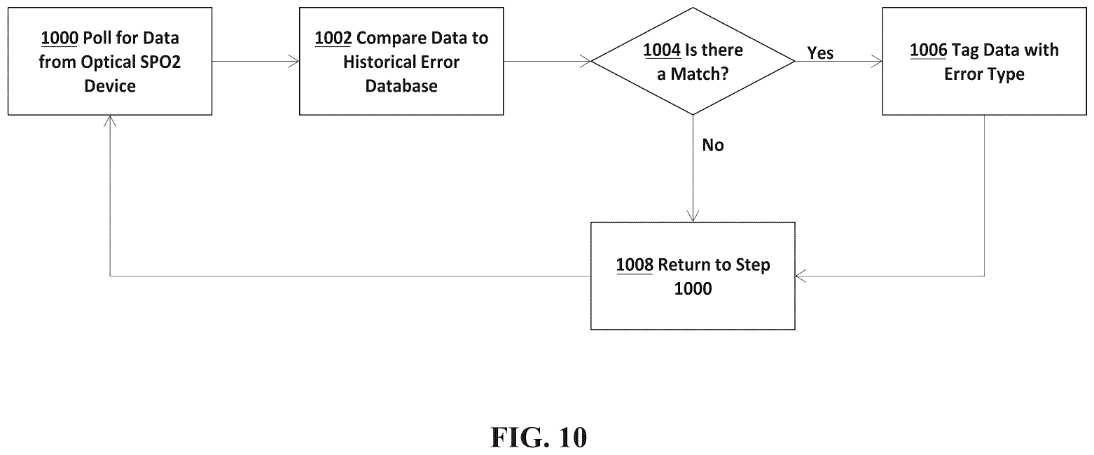

FIG. 10 illustrates an example operation of the error module 146. The error module 146 may poll, at step 1000, for data from the optical SPO2 device 142. Since it may be difficult to assess error in a single data point, the error module 146 may not move onto step 1002 until there is enough data to comprise a waveform. For example, the error module 146 may poll and collect data for 10 seconds before moving to step 1002 so that there are at least 10 seconds of data to search for errors. The error module 146 may compare, at step 1002, the waveform recordings from the optical SPO2 device 142 to waveforms in the historical error database 148. The error module 146 may determine, at step 1004, if there is a match between the waveform from the optical SPO2 device 142 and the error waveforms in the historical error database 148. Matching may involve convolution and/or cross-correlation of the waveforms or any other matching technique. Cross-correlation and convolution are mathematical operations that can be used to determine the similarity between two wave functions. They are often used in signal processing and image recognition applications to find patterns or features in data. Cross-correlation is a measure of the similarity between two signals as a function of the time lag applied to one of them. It is defined as the integral of the product of two signals after one is flipped and delayed by some amount. By running the cross-correlation function on two wave functions, the output will give a similarity value between two signals, where the highest value represents the most similar pair. Convolution, on the other hand, is a mathematical operation that combines two functions to produce a third function. It is the integral of the product of two functions after one of them is flipped and then shifted. By applying convolution on two wave functions, the output will be a function whose values represent the degree of similarity between input signals, where higher values represent more similar signals. These operations may be used in combination and/or with other techniques, such as Fourier transform, to extract information from signals and compare them. Matching waveforms may be waveforms where the cross-correlation and/or convolution values are close to 1 with respect to time. For example, the threshold value may be 0.85. Any point in the function that results from cross-correlation that is above 0.85 may indicate that the waveform matches the error waveform. If there is a match, the error module 146 may tag, at step 1006, the waveform data with the corresponding error type. This tag may be a simple string such as “motion error” or “skin pigmentation error” and may be data or metadata associated with the waveform data. When data is sent from the optical SPO2 device 142 to the connection module 134, it may include this tag. If no matching error waveforms exist, the error module 146 may skip to step 1008. The error module 146 may then return, at step 1008, to step 1000 to continue polling for data from the optical SPO2 device 142.

In one example, a method for measuring analytes using a real-time, non-invasive RF analyte detection device can include providing the real-time, non-invasive RF analyte detection device; providing a non-invasive optical SPO2 device; collecting data from the real-time, non-invasive RF analyte detection device and the non-invasive optical SPO2 device; executing a fusion module to combine the data collected from the real-time, non-invasive RF analyte detection device and the non-invasive optical SPO2 device to generate fused data results; and reporting the fused data results.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A health monitoring system, comprising:
a non-invasive radio-frequency (RF) analyte detection device that is configured to non-invasively detect an analyte in a user; the non-invasive RF analyte detection device having a transmit antenna and a receive antenna, the transmit antenna is positioned and arranged to transmit RF signals into the user, and the receive antenna is positioned and arranged to detect RF response signals resulting from transmission of the RF signals by the transmit antenna into the user;
a non-invasive optical SPO2 device that is configured to non-invasively, optically detect oxygen saturation levels in the user;
a database in communication with the non-invasive RF analyte detection device and with the non-invasive optical SPO2 device, the database storing data obtained by the non-invasive RF analyte detection device and storing data obtained by the non-invasive optical SPO2 device;
a fusion module in communication with the database, the fusion module is configured to select data stored in the database from the non-invasive RF analyte detection device and from the non-invasive optical SPO2 device, and fuse the selected data.

2. The health monitoring system of claim 1, wherein the analyte detected by the non-invasive RF analyte detection device comprises oxygen.

3. The health monitoring system of claim 1, further comprising a fusion example database that contains a plurality of sets of example data, each set of example data includes example data from the non-invasive RF analyte detection device and example data from the non-invasive optical SPO2 device.

4. A health monitoring method, comprising:
collecting analyte data from a user using a non-invasive radio-frequency (RF) analyte detection device that is configured to non-invasively detect an analyte in the user by transmitting RF signals from a transmit antenna into the user and detecting, using a receive antenna, RF response signals resulting from transmission of the RF signals by the transmit antenna into the user;
collecting oxygen saturation level data on oxygen saturation levels of the user using a non-invasive optical SPO2 device that is configured to non-invasively, optically detect the oxygen saturation levels;
storing the analyte data and the oxygen saturation level data in a database that is in communication with the non-invasive RF analyte detection device and with the non-invasive optical SPO2 device;
selecting analyte data and oxygen saturation level data from the database;
executing a fusion module to fuse the selected analyte data and the selected oxygen saturation level data to generate fused data results; and
reporting the fused data results.

5. The health monitoring method of claim 4, wherein the analyte data is oxygen data.
